(19) United States
(12) Patent Application Publication
(10) Pub. No.: US 2025/0114620 A1
(43) Pub. Date: Apr. 10, 2025

(54) ANATOMICALLY INFORMED CLINICAL EFFECT MAPS IN APPLICATION OF DEEP BRAIN STIMULATION

(71) Applicant: Boston Scientific Neuromodulation Corporation, Valencia, CA (US)

(72) Inventors: Mahsa Malekmohammadi, Sherman Oaks, CA (US); Lisa Denise Moore, Glendale, CA (US)

(73) Assignee: Boston Scientific Neuromodulation Corporation, Valencia, CA (US)

(21) Appl. No.: 18/905,684

(22) Filed: Oct. 3, 2024

Related U.S. Application Data
(60) Provisional application No. 63/542,677, filed on Oct. 5, 2023.

(51) Int. Cl. A61N 1/372; A61N 1/05

(52) U.S. Cl. CPC A61N 1/37241; A61N 1/0534; A61N 1/37247

(57) ABSTRACT

Methods and systems for pre-populating clinical effects maps for use in determining therapy parameters for a patient having a neuromodulation system. Neuromodulation planning may be aided by generating a map comparing electrode position and amplitudes used as a tool to test therapies on a patient and determine position and amplitude combinations associated with side effects and clinical benefits. The map may be pre-populated with borders indicating likely combinations for generating side effects and/or clinical benefits. The pre-populated map may be generated using the patient's anatomical data and data stored in a database.

### CROSS-REFERENCE TO RELATED APPLICATIONS

This application claims the benefit of U.S. Provisional Patent Application Ser. No. 63/542,677, filed Oct. 5, 2023, which is incorporated herein by reference.

### BACKGROUND

Deep brain stimulation (DBS) involves the use of a pulse generator attached to the proximal end of a lead, the distal end of the lead being positioned inside the head of a patient. Neuromodulation can be achieved by, for example, issuing therapy pulses to the sub-thalamic nucleus or other neural structure in the brain to provide various forms of treatment. DBS may, for example, reduce tremor for a patient having Parkinson's disease. Other neural structures and conditions may be treated, with a number of potential therapies under development or clinical research and study, such as for cognitive disorders, tremors, Alzheimer's, depression and other diseases.

Modern systems have a plurality of electrodes on the lead allowing spatial selection of structures to receive therapy pulses. A lead may have segmented electrodes about its perimeter to allow such spatial control. This may be called directional therapy and may use current or voltage steering with multiple current or voltage sources. Various other parameters (repetition rate, pulse width/duty cycle, and amplitude, among others) can be modified. The physician may have thousands of options for therapy delivery.

The existing standard of care for DBS includes a significant amount of trial and error to determine, in a fitting process for a given patient, parameters that provide the clinical benefits and side effects of neuromodulation when delivered to the brain. Other neuromodulation implants, such as spinal cord, occipital nerve, sacral nerve, and peripheral nerve modulation systems likewise rely on trial and error to determine benefit and side effects of stimulation delivered at different positions, amplitude and/or other parameters. New and alternative methods and systems for streamlining the fitting process are desired.

### OVERVIEW

The present inventors have recognized, among other things, that a problem to be solved is the need for new and/or alternative methods and systems for streamlining the fitting process are desired. In particular, the use of data obtained from a population of patients, or other comparable devices, may be used to pre-populate a clinical effects map that is developed and tailored to the individual patient during the fitting process.

A first illustrative and non-limiting example takes the form of a method of creating a clinical effects map for a patient having an implantable neuromodulation lead implanted at a location near neural structures, the lead having a plurality of electrodes thereon and being coupled to a pulse generator, the pulse generator in communication with a programmer, the method comprising: obtaining imaging information including the location of the lead and one or more neural structures near the lead; creating a map of electrode positions; placing borders of regions of likely therapy benefit and likely side effects on the map of electrode positions; displaying the map with borders to a user via the programmer; receiving, at the programmer, a user selection of a location to test neuromodulation therapy; issuing from the programmer an instruction to the pulse generator to generate the test neuromodulation therapy at the location; receiving, at the programmer, an indication of whether the test neuromodulation therapy caused a therapy benefit, a side effect, or no effect; and updating the borders on the map responsive to the indication having a therapy benefit or a side effect.

Additionally or alternatively, the borders of regions of likely therapy benefit include a first border and a second border, the first border indicating a first likelihood of therapy benefit occurring in response to therapy pulses within the first border, and the second border indicating a second likelihood higher than the first likelihood of therapy benefit occurring in response to therapy pulses within the second border.

Additionally or alternatively, the borders of regions of likely side effects include a first border and a second border, the first border indicating a first likelihood of side effects occurring in response to therapy pulses within the first border, and the second border indicating a second likelihood higher than the first likelihood of neural side effects occurring in response to therapy pulses within the second border.

Additionally or alternatively, the map displays electrode position along the lead on a first axis, and pulse amplitude on a second axis. Additionally or alternatively, the borders include: a first side effect border determined from the imaging information; and the first side effect border forms an open region having a lowest amplitude and encompassing all amplitudes higher than the lowest amplitude. Additionally or alternatively, the borders include: a first benefit border determined from the imaging information; and the first benefit border forms an enclosed region within a range of amplitudes having upper and lower limits. Additionally or alternatively, the borders include: a first benefit border determined from the imaging information; a first side effect border determined from the imaging information; the first benefit border forms an enclosed region within a range of amplitudes having upper and lower limits; and the first side effect border forms an open region having a lowest amplitude and encompassing all amplitudes higher than the lowest amplitude. Additionally or alternatively, the user interface allows rotation of the map view.

Additionally or alternatively, the step of receiving, at the programmer, a user selection of a location to test neuromodulation therapy includes each of receiving an indication of which electrodes on the lead to use, and an amplitude to test.

Additionally or alternatively, the step of updating the borders on the map occurs automatically in response to the indication.

Additionally or alternatively, the lead is implanted in the brain of the patient, and the neural structures are in the brain of the patient.

Another illustrative and non-limiting example takes the form of a programmer for use in a neuromodulation system, the programmer configured for use with an implantable system having an implantable neuromodulation lead implanted in a patient at a location near neural structures, the lead having a plurality of electrodes thereon and being coupled to a pulse generator, the pulse generator in communication with the programmer, the programmer having a processor, a user interface, and machine readable instructions for the programmer to perform the following: obtaining imaging information including a location of the lead in the patient and a location of one or more neural structures near the lead; creating a map of electrode positions; placing borders of regions of likely therapy benefit and likely side effects derived using the imaging information on the map of electrode positions; a) displaying the map with borders to a user via the user interface; receiving, at the user interface, a user selection of a test location to test neuromodulation therapy; issuing an instruction to the pulse generator to generate the test neuromodulation therapy at the test location; receiving, at the programmer, an indication of whether the test neuromodulation therapy caused a therapy benefit, a side effect, or no effect; updating the borders on the map responsive to the indication having a therapy benefit or a side effect; and b) displaying the map with the updated borders to the user via the user interface.

Additionally or alternatively, the borders of regions of likely therapy benefit include a first border and a second border, the first border indicating a first likelihood of therapy benefit occurring in response to therapy pulses within the first border, and the second border indicating a second likelihood higher than the first likelihood of therapy benefit occurring in response to therapy pulses within the second border.

Additionally or alternatively, the borders of regions of likely side effects include a first border and a second border, the first border indicating a first likelihood of side effects occurring in response to therapy pulses within the first border, and the second border indicating a second likelihood higher than the first likelihood of neural side effects occurring in response to therapy pulses within the second border.

Additionally or alternatively, the map displays electrode position along the lead on a first axis, and pulse amplitude on a second axis. Additionally or alternatively, the borders include a first side effect border; and the first side effect border forms an open region having a lowest amplitude and encompassing all amplitudes higher than the lowest amplitude. Additionally or alternatively, the borders include a first benefit border; and the first benefit border forms an enclosed region within a range of amplitudes having upper and lower limits.

Additionally or alternatively, the borders at a) include: a first side effect border indicating a first likelihood of a side effect; a second side effect border indicating a second likelihood of a side effect higher than the first likelihood of a side effect; and the updated borders at b) replace the first side effect border and the second side effect border with a third side effect border indicating a side effect occurred during testing.

Additionally or alternatively, the machine readable instructions for the programmer cause the processor to receive, at the programmer, a user selection of a location to test neuromodulation therapy, by each of receiving an indication of which electrodes on the lead to use, and an amplitude to test.

Additionally or alternatively, the processor automatically performs the step of updating the borders on the map in response to the indication.

Additionally or alternatively, the neural structures are in the brain of the patient. Additionally or alternatively, the neural structures are in the spinal column of the patient. Additionally or alternatively, the neural structures are near the Vagus nerve of the patient. Additionally or alternatively, the neural structures are near the sacral nerve of the patient.

Another illustrative and non-limiting example takes the form of a system comprising: an implantable lead; an implantable pulse generator having operational circuitry therein for generating electrical outputs, and adapted to receive the lead such that the operational circuitry can issue the electrical outputs to a patient via the implantable lead; and a programmer having a processor, a user interface, and machine-readable instructions for the programmer to perform the following: obtaining imaging information including a location of the lead in the patient and a location of one or more neural structures near the lead; creating a map of electrode positions; placing borders of regions of likely therapy benefit and likely side effects derived using the imaging information on the map of electrode positions; displaying the map with borders to a user via the user interface; receiving, at the user interface, a user selection of a test location to test neuromodulation therapy; issuing an instruction to the pulse generator to generate the test neuromodulation therapy at the test location; receiving, at the programmer, an indication of whether the test neuromodulation therapy caused a therapy benefit, a side effect, or no effect; updating the borders on the map responsive to the indication indicating a therapy benefit or a side effect; and displaying the map with the updated borders to the user via the user interface.

Additionally or alternatively, the lead and implantable pulse generator form a Deep Brain Stimulation system.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 show an illustrative DBS system in a patient;

FIG. 2 shows a directional lead in relation to neural structures associated with clinical benefits and side effects;

FIGS. 3A-3C illustrate a process of populating a clinical effects map;

FIGS. 4A-4C show several features for pre-populating a clinical effects map;

FIGS. 5A-5B show an illustrative example; and

FIG. 6 shows an illustrative method in block flow form.

### DETAILED DESCRIPTION

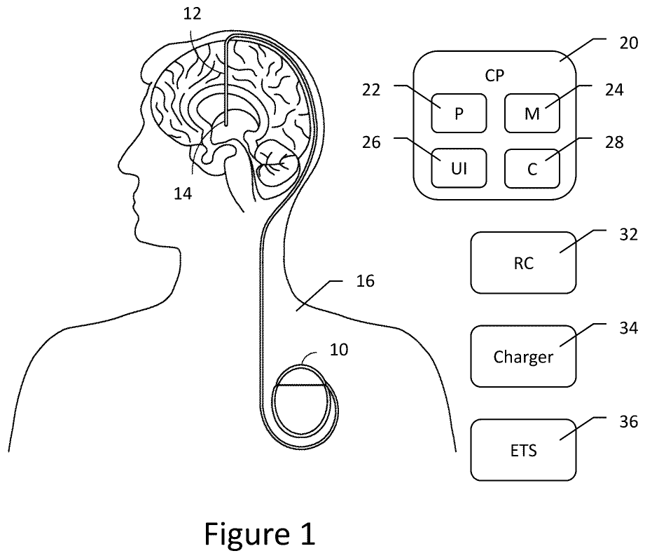

FIG. 1 shows an illustrative DBS system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead.

The lead 12 may be placed at any suitable location of the brain where a target for therapy is identified. For example, a lead 12 may be positioned so that the distal end 14 is near the mid-brain and/or various structures therein that are known in the art for use in providing neuromodulation to treat various diseases. A lead used in DBS may include a combination of segmented and ring electrodes, if desired, such as disclosed in U.S. Pat. Nos. 8,483,237 and 8,321,025, the disclosures of which are incorporated herein by reference.

The IPG 10 may include separate circuits, sometimes referred to as operational circuitry, including a microcontroller (which may also be implemented as part of a microprocessor if desired), which controls operations of the IPG at a high level. The IPG can include a power source, typically a battery (rechargeable or primary cell, as desired), though some systems may be adapted to operate without a battery by receiving power inductively or through other link (such as radiofrequency) and issuing therapy using the received power without long-term storage. The microcontroller may include memory for storing operational instructions in a non-transitory media, such as a Flash memory, RAM, ROM, etc. The IPG includes stimulation circuitry. At a high level the stimulation circuitry may include a plurality of current sources and current sinks (for a current-controlled system; a plurality of voltage sources may be used in voltage-controlled systems instead), and control circuitry including for example one or more analog ASICs, as well as switch arrays that implement steering instructions and/or electrode selections. U.S. Pat. No. 10,716,932 provides illustrative details for both current and planned future implementations of the stimulation circuitry, and is incorporated herein by reference.

The IPG 10 may include a conductive outer housing that can serve as a return electrode or indifferent electrode during therapy delivery, as desired. A header provides feedthrough circuitry allowing the IPG 10 to couple to a lead 12, with separate electrical connections to each of the electrodes. The IPG header and housing provide a hermetic sealed environment for the operational circuitry. In some examples, a plurality of programs can be set for therapy delivery by the IPG 10. Each program may operate according to a schedule and individual program parameters.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system described below includes various external devices. A clinician programmer (CP) 20 may be used to determine/select therapy programs. The CP 20 can be used by a physician to manipulate the outputs of the IPG 10 and/or an external test stimulator (ETS) 36. For example, the CP 20 can be used by the physician to define a therapy regimen or program for application to the patient. Multiple programs may be facilitated and stored by the IPG 10 or ETS 36; in some examples, a patient remote control (RC) 32 may store the programs to be used.

The CP 20 may be, for example and without limitation, a computer such as a laptop or tablet computer. The CP 20 therefore includes a microcontroller and/or microprocessor, shown as processor 22 and associated memory 24. The memory 24 may take any suitable form (RAM, ROM, Flash, etc.), and stores machine readable instructions allowing the processor 22 to perform the methods disclosed herein. To the extent Bluetooth is used as a communications protocol, the RF circuitry may be included in the device as a communications circuitry 28, located internal to the CP 20. If some other communications technology (inductive or Medradio) is used, or if range is limited by the IPG for example, the communications circuit 28 may be provided via a wand having specialized circuitry (for Bluetooth, Medradio, or inductive telemetry) therein that couples, for example, to a USB port on the CP. The CP will include a user interface 26, such as a screen or touchscreen, keyboard, mouse, trackball, etc. allowing the user to provide instructions and make choices. The RC may take the form of a dedicated device, a locked off-the shelf device (such as a smartphone) or may be a multi-use device such as a smartphone running an app.

The CP 20 may be used to determine stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 20 can be used by a physician, or at the direction of a physician, to obtain data from and provide instructions the IPG 10 via suitable communications protocols such as Bluetooth or MedRadio or other wireless communications standards, and/or via other modalities such as inductive telemetry.

The RC 32 can be used by the patient to perform various actions relative to the IPG 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 20 to control and/or obtain data from the IPG 10. The patient RC 32 may also be programmable on its own, or may communicate or be linked with the CP 20. The RC 32 may be a dedicated device, including a custom device, a locked off-the-shelf device with specialized software to prevent other uses, or may be a multi-purpose device such as the patient's cell phone.

A charger 34 may be provided to the patient to allow the patient to recharge the IPG 10, if the IPG 10 is rechargeable. Some IPG 10 are not rechargeable, and so the charger 34 may be omitted. The charger 34 can operate, for example, by generating a varying magnetic field to activate an inductor associated with the IPG 10 to provide power to recharge the IPG battery, using known methods and circuitry.

Some systems may include the ETS 36. The ETS 36 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 36, and the ETS 36 can be programmed using the CP 20 with various therapy programs and stimulation parameters. Once therapy suitability for the patient is established to the satisfaction of the patient 16 and/or physician, the permanent IPG 10 is implanted and the lead 12 is connected thereto, with the ETS 36 then removed from use. Additional components, such as a remote monitoring system or bedside monitor (not shown) may also be included.

The figures and explanations herein focus primarily on use of the present invention for DBS purposes. That said, a clinical effects map and the related processes that are disclosed may also be used in other neural therapy systems, including any of Vagus nerve stimulation (VSN), spinal cord stimulation (SCS), occipital nerve therapy, sacral nerve therapy, etc.

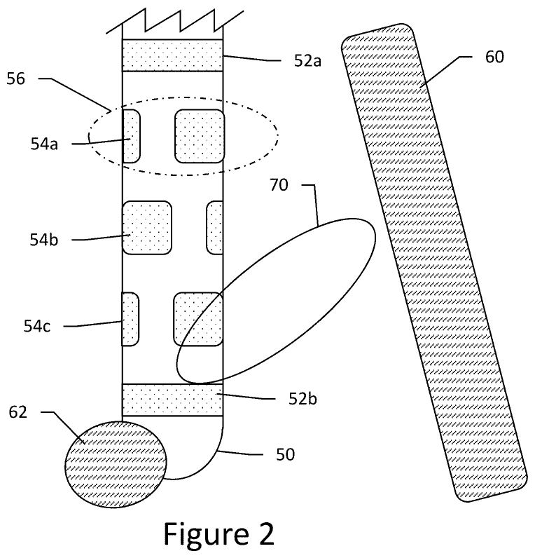

FIG. 2 shows a directional lead in relation to neural structures associated with clinical benefits and side effects. The lead has a distal end 50 and carries two ring electrodes 52a, 52b (collectively, ring electrodes 52), and a number of segmented electrodes 54a, 54b, 54c (collectively, segmented electrodes 54). Each electrode 52, 54 can be separately addressed using multichannel outputs, which can be voltage controlled or current controlled. Some systems may use multiple independent current control, as is known in the art.

As an example of a directional or spatially selective therapy, the use of only the electrodes level with electrode 54a may generate an electrical field that may be visualized as shown at 56, where the outer border of 56 represents an equipotential or equal-field line relative to a voltage or current controlled output using a distant or remote electrode, such as the housing of the pulse generator (not shown).

Neural structures are identified in relation to the lead 50. For example, avoid structures are illustrated at 60, 62, representing neural tissue that, if stimulated, may cause a side effect to the patient. Structures associated with clinical benefits, such as that shown at 70, may also be present in the vicinity of the lead 50. With the understanding that a DBS lead can positioned relative to neural structures associated with clinical benefits as well as neural structures associated with side effects, and further noting DBS therapy may be tailored to a particular patient, the programming of such a lead is important to therapy outcomes. It should also be noted that when programming for DBS, it is also desirable both for power consumption purposes (linked to battery longevity) and to avoid triggering new, unknown side effects, to limit the volume of tissue that is subject to electrical fields.

Using the visual representation of FIG. 2 to explain the aims and complexities, it is desirable to deliver therapy to region 70, without causing stimulus in regions 60 and 62, while also minimizing the total volume of tissue that is subject to stimulation. However, when the lead is positioned in the brain, there is limited capability for understanding the exact borders of any of structures 60, 62, and 70 based solely on patient specific imaging, brain atlas data, and/or patient population data. Further, it may not be known how much stimulation of any such structures will cause therapeutic benefits and/or side effects, and the amplitude, intensity, frequency, pulse width, and/or pulse shape/type that will cause either benefits or side effects may vary from one patient to another.

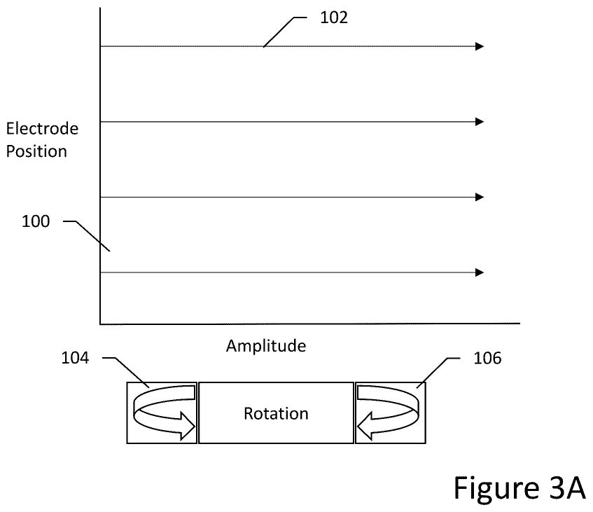

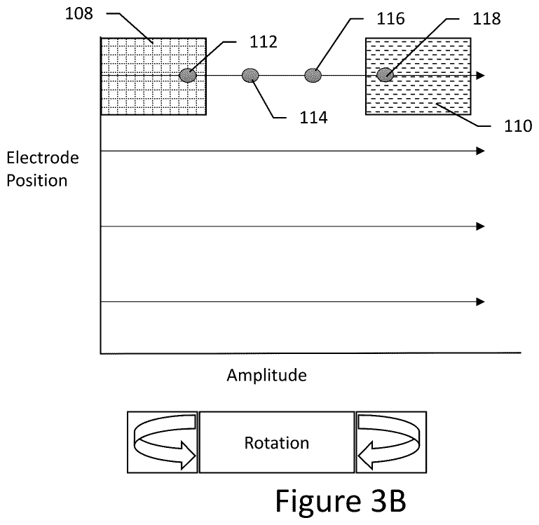

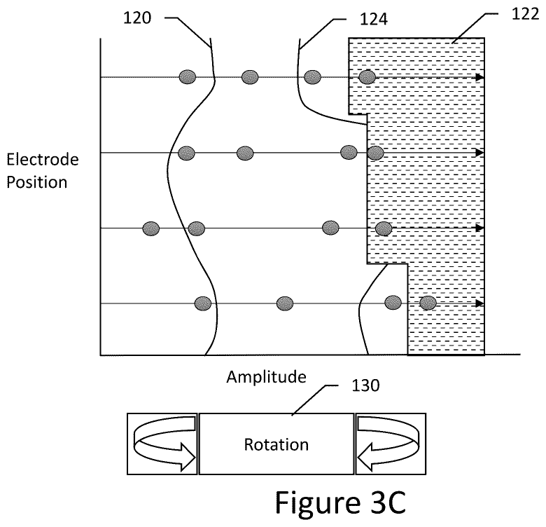

A clinical effect map is utilized to create an understanding of how stimulation at various lead electrodes with different strength treat patient. It illustrates the interaction of stimulation at different levels of the lead (vertical axis) at various stimulation intensities (i.e., amplitudes) and benefit and side effects inducted in the patient. FIGS. 3A-3C illustrate a process of populating a clinical effects map. To address the above noted limitations on precise knowledge of brain structures, as well as variation across patients, some degree of patient testing and trial and error is typically needed. The clinical effects map has been developed as one tool for the patient testing process. A clinical effects map, as previously used, is shown in FIG. 3A and starts with a blank template 100 with electrode position on one axis and amplitude on the other axis. Each horizontal line 102 may indicate a level at which electrodes reside on the lead; a 1:1 correspondence is not needed for the horizontal lines to actual electrodes, and the lines 102 may indicate halfway points between electrode edges, centers, etc., as desired. The map view may be rotated using icons 104, 106, in keeping with the use of a cylindrical and directional lead as indicated in FIG. 2. This map, as well as the other clinical effects maps shown below, may be displayed in the user interface 26, as determined by the processor 22 using instructions from memory 24, in the CP 20 (FIG. 1).

As shown in FIG. 3B, testing is performed by issuing (optionally monopolar) stimulation using electrodes at a selected electrode position or level, with varying stimulation strength or amplitude. The process defines regions of no clinical benefit 108 (generally corresponding to therapy strength/amplitude being too low), regions where there are one or more clinical benefits observed (typically with varying degree of benefit based on stimulation strength/amplitude) and, finally, side effect regions 110 (where therapy amplitude is high enough that a side effect is observed). Starting from a relatively low amplitude, tests are performed as indicated at 112, 114, 116, and 118. The region of no clinical benefit 108 extends over point 112, indicating that the amplitude tested at 112 was too low to provide therapeutic benefit, but did not cause a side effect. Subsequent iterations of testing at 114, 116, 118 are represented on the map with the dots as shown. The region no clinical benefit does not extend to 114, because testing at that amplitude had a therapeutic benefit or effect, but not a side effect. When the amplitude gets higher, as shown at 118 in the example, a side effect is observed, and so the side effect region 110 extends over dot 118.

FIG. 3C shows the result after several rounds of testing at different electrode positions and amplitudes. The region of no clinical benefit 104 is now illustrated as a low amplitude border 120, below which therapy output is considered ineffective. The side effect region is now shown at 122. Depending on the severity of side effects, for example, additional boundaries may be included as indicated at 124, if desired. While FIG. 3C shows the results in a black and white drawings, a heat map may be used with one color representing the side effect region, and a second color varying in intensity/brightness across the rest of the clinical effects map, with varying intensity or brightness indicating predictions of higher likelihood and lower likelihood of therapeutic benefits.

What may be noted in FIG. 3C is that there may be a large number of tests required to create a complete map, and this may still leave regions of uncertainty unless still further tests are performed. For example, a number of test points are shown already in FIG. 3C, however, rotation 130 has not yet been considered, and additional testing may be performed after rotating the view to account for the directional nature of the lead which may have segmented electrodes (see FIG. 2) allowing targeting at various rotational angles relative to that shown in FIG. 3C. The testing is time consuming for both the clinician and the patient and eliciting side effects is especially uncomfortable to the patient. It is desirable in some illustrative examples to pre-populate the clinical effects map so that a faster and more streamlined testing approach can be used.

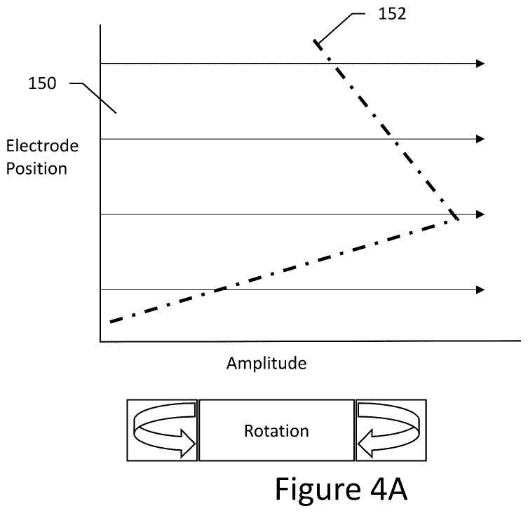

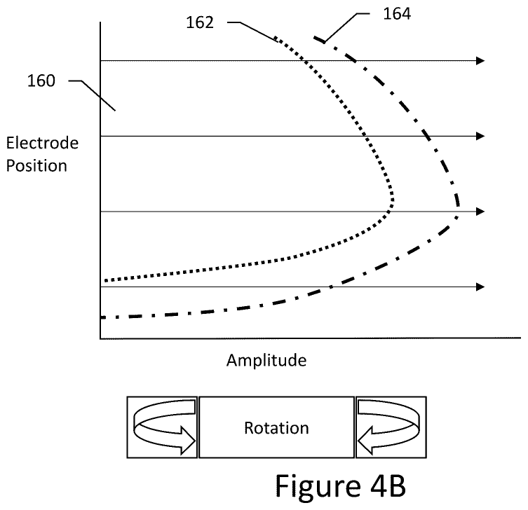

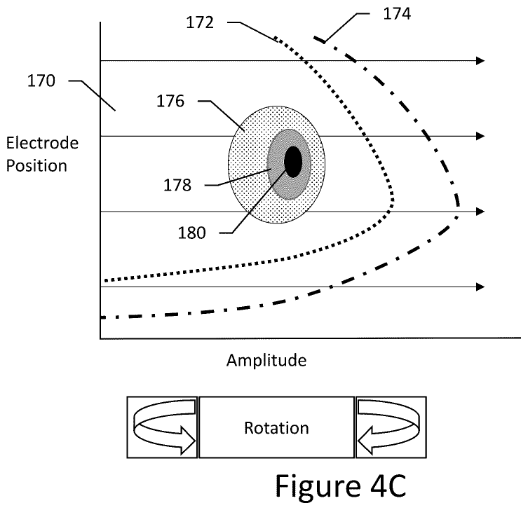

FIGS. 4A-4C show several features for pre-populating a clinical effects map. As noted previously, there are many sources of data available to the system. Population based data, informed by known features of interaction between stimulation and various brains structures and derived from healthy persons and patients having similar conditions to the patient being treated, patients who have previously received an implanted system having a lead, and the like, may be useful sources of data. In addition, the patient in whom a DBS system is implanted will typically undergo pre-operative imaging, such as with MRI (structural and/or functional), and one or more scans may take place as the lead is introduced in the patient's brain as well as post-operative imaging which may also be taken. These various sources can be used to estimate the position of structures in the patient's brain associated with clinical benefits and side effects, in relation to the lead. As shown in FIG. 4A, for example, such information may be used to estimate a border 152 on a clinical map 150 at which side effects may be expected to occur. The border 152, is not directly an indication of a spatial location, but instead estimates an amplitude at which therapeutic pulses may be expected to cause a side effect. Having borders of this type available in the clinical effects map may be useful to streamline the testing process.

FIG. 4B shows another example. In this version of a clinical effect map 160, a first border is shown at 162 as a minimum amplitude at which a side effect is deemed likely to arise, and a second border 164 is intended to indicate a likelihood above a threshold that a side effect is likely. For example, border 162 may represent a 20% likelihood, in view of data obtained as explained below, of a side effect at the given electrode position and amplitude. Border 164 may represent an 80% likelihood of a side effect in a similar way. Borders 162 and 164 are shown in simplified form. Depending on location in the neural tissue there may be multiple benefit or side effect structures near the lead, making for more complex borders and/or the possibility of multiple borders, islands, etc. within the pre-populated clinical effects map.

FIG. 4C shows another example. Here, a benefit region is pre-populated on a clinical effects map 170. As before, first and second borders are shown at 172, 174 for a side effect region. A benefit structure is also identified, illustratively with plural borders at 176, 178, 180, each with increasing likelihood of therapeutic benefit identified (such as 20%, 50%, 80%).

Rather than discrete lines as shown, gradients may be used in a color scheme for representing increasing likelihood of finding a location and amplitude of greater or lesser clinical benefit and/or side effects.

The borders shown may be derived by identifying structures known to cause side effects in a clinical population of other patients, and or structures known to cause therapeutic benefits in a clinical population of other patients; such structures may be estimated as well using non-clinical population data (healthy persons). Knowing which structures to avoid or benefit, the next step can be to determine from the lead position, as indicated in post-operative imaging (or intraoperative imaging), where the lead is in relation to patient-specific landmarks. Then, patient imaging, such as a structural MRI, may be aligned with the lead location information, where the structural MRI may be useful to indicate where the structures of interest are likely found (structures associated with clinical benefits or structures associated with side effects). All borders may be considered imprecise in this analysis, and so gradients of likelihood can be used.

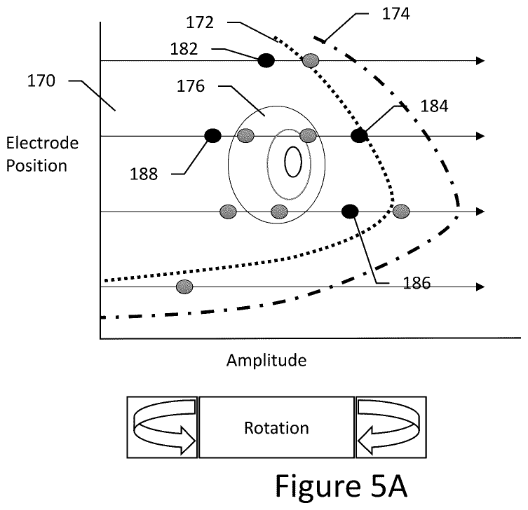

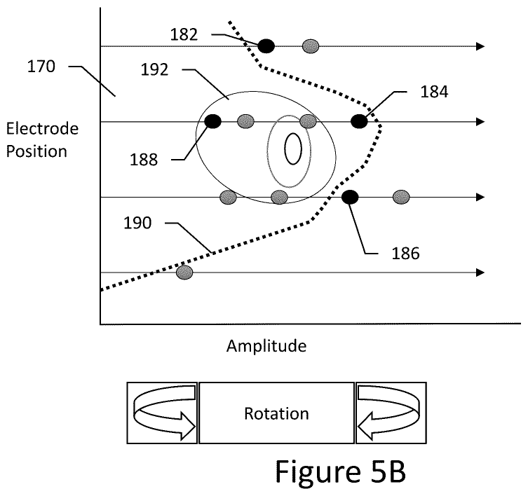

FIGS. 5A-5B show an illustrative example for use of a clinical effect map that is pre-populated with data related to clinical benefit and/or side effect structures. Referring to FIG. 5A, the same clinical effects map 170 as shown above in FIG. 4C is again illustrated, with borders at 172 and 174 related to the likelihood of a side effect, and borders for a region of likely clinical benefit also shown in at 176. A number of test therapies have been performed in this example, including for example, those highlighted in black at 182, 184, 186 and 188. As can be seen, therapy testing is focused to some extent on the locations of the pre-populated boundaries. It should be noted that the use of grey and black fill in the indications of testing positions/amplitudes is not a coding of whether side effects, benefits, or no response were observed.

In the example, location 182 was not predicted by the prepopulated data to generate a side effect, as it lies outside of the region bounded by border 172. However, in this prophetic example, the testing found a side effect when pulses are delivered at the amplitude and electrode positions represented at 182 and 186. Meanwhile, location 184 is to the right of border 172, but did not yield a side effect. Finally, the heightened clinical benefit for region 176 was also found during testing at the location and amplitude represented by dot 188.

In response to testing, the clinical effect map is updated as shown in FIG. 5B, with border 172 redrawn as border 190. Border 174 has been removed, as no longer necessary (if a gradient or color-coded mapping is used, such removal would instead show up as coloration indicating a high side effects anywhere to the right of border 190). Also, the border surrounding the “sweet spot” or location of significant clinical benefit is also adjusted to encompass location 188. Testing can thus be performed with less guesswork and may be streamlined to require less time. Border adjustment may be a manual process or may be automated by the system using the testing data and, for example, a smoothing function to adjust the border positions.

Some characteristics of the resulting map may be noted. The initial set of borders in FIG. 5A had multiple borders for the side effects indicating different likelihoods. Such multiple shells may be included in the final clinical effects map if desired.

The example of FIG. 5B, on the other hand, only shows a single side effect border 190, reflecting the testing having been performed to replace likelihoods with tested results. It may be the case, in some examples, that multiple different side effects, possibly with differing severity or resulting from activation of different side effect structures, are found during testing, in which case there may be multiple borders instead of a single border 190. Further, the side effects border in the mapping as shown has a minimum amplitude above which side effects may be expected to occur; relative to therapy planning, the amplitudes at which line 190 resides may be understood as indicating a maximum amplitude at the given electrode position.

The clinical benefits or sweet spot region at 192, on the other hand, may still include multiple internal borders if desired, as there may be increased benefits at specific positions and amplitudes. The border 192 of the clinical benefits region is, in the example shown, a closed border on a graph having amplitude and electrode position as axes. This need not be the case, but can be useful to the physician as there is a minimum amplitude below which no therapy benefit (possibly no neural activation) occurs. Thus, the two borders 190 and 192 are different in that one is open (190) and the other is closed (192) in the illustration of FIG. 5.

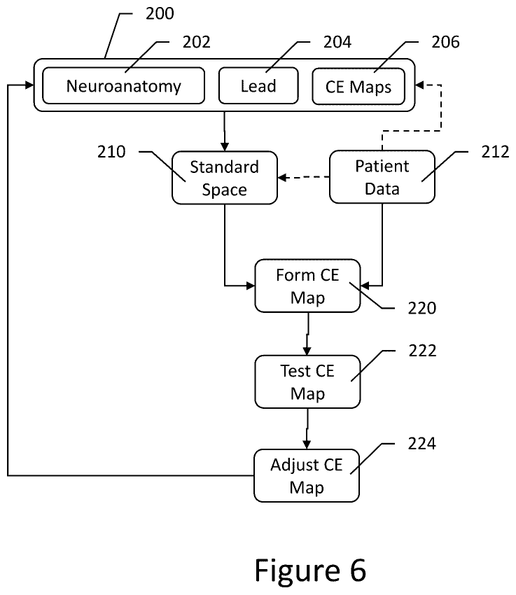

FIG. 6 shows an illustrative method in block flow form. This example may be executed by, for example, one or more of a clinician programmer operating on its own, or a clinician programmer operating in conjunction with a central server and computer system. Block 200 indicates that a database including neuroanatomy data 202, lead positioning data 204, and clinical effects maps 206 may be queried to develop a prepopulated clinical effects map in a standard space, as indicated at 210. The standard space may include, for example, use of any suitable anatomical coordinate system, such as but not limited to the Talairach space and the Montreal Neurological Institute space, as desired.

Patient data, such as from MRI, CT, X-Rays, or any other imaging modality, is also obtained as indicated at 212. These data sets are combined together at 220. This step of combining the data may be performed for example, by co-registration of landmarks in the data sets, and overlapping of population-based data from the standard space 210 with the patient imaging data 212 to generate the estimated positioning of side effect and benefit structures in the patient's neural anatomy, as illustrated above for example, in FIGS. 4A-4C. The physician's input may be used as well to identify the benefit and side effect structures that the physician wishes to see on the clinical effects map (“CE Map” in the Figure).

Testing is then performed using the CE Map, as indicated at 222. Testing may focus, for example and subject to clinical judgment, on particular regions of interest, such as the boundaries represented on the CE Map that may indicate location and amplitude combinations likely to induce side effects and/or therapeutic benefits. To the extent that the map formed at 220 turns out to be inaccurate relative to the patient's actual response, the map can be adjusted at 224. For example, if a therapy benefit or a side effect occurs during the testing at a location on the map that does not indicate likely benefit or side effect, then the CE map can be adjusted. If no effect is observed at a particular location, this may or may not lead to adjustment of the CE map. In some examples, the therapy map can be adjusted even if the therapy delivered in 222 causes a predicted effect (no effect, side effect, or therapy benefit) by modifying the border on the amp from being a likelihood to being a tested result, if desired.

The final CE Map may be stored as part of the patient's medical records, such as in a central server, or on a CP, an RC, or even in the memory of the patient's IPG, as desired. In addition, the CE Map from 224 may be communicated to the database at 200 to provide further tailoring of the data stored therein. For example, gradients or boundaries of likelihood that are generated in the standard space may be based on CE Maps 206. Such updates to the stored set of CE Maps at 206 may, for example, be averaged to generate an “average” CE Map across a population of patients. Updates may also be issued to, for example, a database of lead placements indicated at 204 and/or neuroanatomy data at 202, as desired.

In an example, CE Maps 206 may be grouped into clusters based on anatomical, disease state, age, gender, or other similarities shared among a group of patients. As indicated by the broken line arrows from 212 to 200 and/or 210, the patient data 212 may be used to select “most relevant” data from the database 200. For example, one or several characteristics of patients (head size, lead position, disease type, age, etc.) may be communicated in the process to determine which CE Maps 206 or other data sets are a best fit to the patient.

Several elements of the prior art can be explained and placed in context with the above discussion. It is known to generate, for a given patient and a given set of therapy parameters, a stimulation field model (SFM). The SFM is a representation, sometimes in three dimensions, of the volume of tissue that will be subjected to electrical field sufficient to cause a neural response. The SFM can be viewed, for example, relative to lead position in the patient, as well as relative to neural structures in the patient near the lead. For example, using patient-specific imaging information, sometimes combined with population-based data, the SFM may be displayed relative to the lead and a range of target (benefit) and avoid (side effect) structures. The SFM is generated based on a set of therapy parameters, and surrounding structures may be based on the patient-specific imaging information and/or population-based data.

Some therapy optimization approaches begin by analytically dividing tissue in the region of the lead into small volumes, sometimes referred to as voxels. Each voxel can be analyzed further to determine a threshold excitation or activation current for the voxel. The neural structures in the region of the lead can be identified and presented to a physician to identify target (clinical benefit) and avoid (side effect) structures or regions, and each of the voxels can be characterized by their location inside of or outside of the target and avoid structures. Proposed therapy settings may be analyzed using these inputs to determine an optimal therapy (including any of steering, current fractionalization, amplitude, pulse width and/or pulse repetition rate) by weighting the number activated voxels in each of target and avoid volumes, and also weighting total number of activated voxels in an analysis, such as using a cost function. Additional characterization may include assessing power use by the system for each proposed therapy setting. Those therapy settings having the “best” scores in accordance with the weight factors, target and avoid stimulation volumes, total volume, and power factors (and/or other factors) may be identified via a search algorithm, and presented to a user as likely therapies to test (sometimes along with the SFM for such therapy settings). Such optimization again occurs toward the end of the fitting procedure. The present concepts, on the other hand, are directed toward obtaining data from the patient's anatomy to determine the clinical effects mapping that can serve as the basis for optimization.

Each of these non-limiting examples can stand on its own, or can be combined in various permutations or combinations with one or more of the other examples. The above detailed description includes references to the accompanying drawings, which form a part of the detailed description. The drawings show, by way of illustration, specific embodiments that may be referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of creating a clinical effects map for a patient having an implantable neuromodulation lead implanted at a location near neural structures, the lead having a plurality of electrodes thereon and being coupled to a pulse generator, the pulse generator in communication with a programmer, the method comprising:
obtaining imaging information including the location of the lead and one or more neural structures near the lead;
creating a map of electrode positions;
placing borders of regions of likely therapy benefit and likely side effects on the map of electrode positions;
displaying the map with borders to a user via the programmer;
receiving, at the programmer, a user selection of a location to test neuromodulation therapy;
issuing from the programmer an instruction to the pulse generator to generate the test neuromodulation therapy at the location;
receiving, at the programmer, an indication of whether the test neuromodulation therapy caused a therapy benefit, a side effect, or no effect; and
updating the borders on the map responsive to the indication having a therapy benefit or a side effect and displaying the map with updated borders to the user.

2. The method of claim 1, wherein the borders of regions of likely therapy benefit include a first border and a second border, the first border indicating a first likelihood of therapy benefit occurring in response to therapy pulses within the first border, and the second border indicating a second likelihood higher than the first likelihood of therapy benefit occurring in response to therapy pulses within the second border.

3. The method of claim 1, wherein the borders of regions of likely side effects include a first border and a second border, the first border indicating a first likelihood of side effects occurring in response to therapy pulses within the first border, and the second border indicating a second likelihood higher than the first likelihood of neural side effects occurring in response to therapy pulses within the second border.

4. The method of claim 1, wherein the map displays electrode position along the lead on a first axis, and pulse amplitude on a second axis.

5. The method of claim 4, wherein the borders include:
a first side effect border determined from the imaging information; and
the first side effect border forms an open region having a lowest amplitude and encompassing all amplitudes higher than the lowest amplitude.

6. The method of claim 4, wherein the borders include:
a first benefit border determined from the imaging information; and
the first benefit border forms an enclosed region within a range of amplitudes having upper and lower limits.

7. The method of claim 4, wherein the borders include:
a first benefit border determined from the imaging information;
a first side effect border determined from the imaging information;
the first benefit border forms an enclosed region within a range of amplitudes having upper and lower limits; and
the first side effect border forms an open region having a lowest amplitude and encompassing all amplitudes higher than the lowest amplitude.

8. The method of claim 1, wherein receiving, at the programmer, a user selection of a location to test neuromodulation therapy includes each of receiving an indication of which electrodes on the lead to use, and an amplitude to test.

9. The method of claim 1, wherein the step of updating the borders on the map occurs automatically in response to the indication.

10. The method of claim 1, wherein the lead is implanted in the brain of the patient, and the neural structures are in the brain of the patient.

11. A programmer for use in a neuromodulation system, the programmer configured for use with an implantable system having an implantable neuromodulation lead implanted in a patient at a location near neural structures, the lead having a plurality of electrodes thereon and being coupled to a pulse generator, the pulse generator in communication with the programmer, the programmer having a processor, a user interface, and machine readable instructions for the programmer to perform the following:
obtaining imaging information including a location of the lead in the patient and a location of one or more neural structures near the lead;
creating a map of electrode positions;
placing borders of regions of likely therapy benefit and likely side effects derived using the imaging information on the map of electrode positions;
a) displaying the map with borders to a user via the user interface;
receiving, at the user interface, a user selection of a test location to test neuromodulation therapy;
issuing an instruction to the pulse generator to generate the test neuromodulation therapy at the test location;
receiving, at the programmer, an indication of whether the test neuromodulation therapy caused a therapy benefit, a side effect, or no effect;
updating the borders on the map responsive to the indication having a therapy benefit or a side effect; and
b) displaying the map with updated borders to the user via the user interface.

12. The programmer of claim 11, wherein the borders of regions of likely therapy benefit include a first border and a second border, the first border indicating a first likelihood of therapy benefit occurring in response to therapy pulses within the first border, and the second border indicating a second likelihood higher than the first likelihood of therapy benefit occurring in response to therapy pulses within the second border.

13. The programmer of claim 11, wherein the borders of regions of likely side effects include a first border and a second border, the first border indicating a first likelihood of side effects occurring in response to therapy pulses within the first border, and the second border indicating a second likelihood higher than the first likelihood of neural side effects occurring in response to therapy pulses within the second border.

14. The programmer of claim 11, wherein the map displays electrode position along the lead on a first axis, and pulse amplitude on a second axis.

15. The programmer of claim 14, wherein:
the borders include a first side effect border; and
the first side effect border forms an open region having a lowest amplitude and encompassing all amplitudes higher than the lowest amplitude.

16. The programmer of claim 14, wherein:
the borders include a first benefit border; and
the first benefit border forms an enclosed region within a range of amplitudes having upper and lower limits.

17. The programmer of claim 14, wherein the user interface allows rotation of the map view.

18. The programmer of claim 11, wherein the borders at a) include:
a first side effect border indicating a first likelihood of a side effect;
a second side effect border indicating a second likelihood of a side effect higher than the first likelihood of a side effect; and
the updated borders at b) replace the first side effect border and the second side effect border with a third side effect border indicating a side effect occurred during testing.

19. The programmer of claim 11, wherein the machine-readable instructions for the programmer cause the processor to receive, at the programmer, a user selection of a location to test neuromodulation therapy, by each of receiving an indication of which electrodes on the lead to use, and an amplitude to test.

20. A system comprising:
an implantable lead;
an implantable pulse generator having operational circuitry therein for generating electrical outputs, and adapted to receive the lead such that the operational circuitry can issue the electrical outputs to a patient via the implantable lead; and
a programmer having a processor, a user interface, and machine-readable instructions for the programmer to perform the following:
obtaining imaging information including a location of the lead in the patient and a location of one or more neural structures near the lead;
creating a map of electrode positions;
placing borders of regions of likely therapy benefit and likely side effects derived using the imaging information on the map of electrode positions;
displaying the map with borders to a user via the user interface;
receiving, at the user interface, a user selection of a test location to test neuromodulation therapy;
issuing an instruction to the pulse generator to generate the test neuromodulation therapy at the test location;
receiving, at the programmer, an indication of whether the test neuromodulation therapy caused a therapy benefit, a side effect, or no effect;
updating the borders on the map responsive to the indication having a therapy benefit or a side effect; and
displaying the map with updated borders to the user via the user interface.
